(19) United States
(12) Patent Application Publication
(10) Pub. No.: US 2023/0119089 A1
(43) Pub. Date: Apr. 20, 2023

(54) METHOD FOR DIFFERENTIATING PLURIPOTENT STEM CELLS INTO UNDERLYING CONNECTIVE TISSUE FIBROBLASTS OF AN EPITHELIUM

(71) Applicants: ADHARA, PARIS (FR); CENTRE D'ETUDE DES CELLULES SOUCHES, CORBEIL ESSONNES (FR); UNIVERSITE D'EVRY VAL D'ESSONNE, EVRY (FR)

(72) Inventors: Gilles LEMAITRE, EVRY-COURCOURONNES (FR); Christine BALDESCHI, EVRY-COURCOURONNES (FR); Sophie DOMINGUES, EVRY-COURCOURONNES (FR)

(21) Appl. No.: 17/905,528

(22) PCT Filed: Mar. 2, 2021
(86) PCT No.: PCT/FR2021/050352

§ 371 Date: Sep. 2, 2022

(30) Foreign Application Priority Data
Mar. 2, 2020 (EP) 20305214.7

(51) Int. Cl. C12N 5/077

(52) U.S. Cl. CPC C12N 5/0656; C12N 2501/11; C12N 2501/115; C12N 2501/155; C12N 2503/02; C12N 2533/50; C12N 2501/33

(57) ABSTRACT

The invention relates to a method for differentiating human pluripotent stem cells into fibroblasts, characterized in that the human pluripotent stem cells are cultured on an adherent system in the presence of a medium that is suitable for culturing fibroblasts and in the absence of feeder cells.

The present invention relates to a method for differentiating pluripotent stem cells (PSCs) (embryonic or induced pluripotent stem cells (iPS)) into fibroblasts of fibrous connective tissue underlying an epithelium.

### CONTEXT OF THE INVENTION

Human pluripotent stem cells, whether embryonic or induced to pluripotency, have the ability to proliferate identically (each mother cell giving rise to two daughter cells identical to the first) indefinitely, without ever senescing as do all other cells of the organism, and the ability, under other culture conditions, to differentiate to give rise to any cell of the organism (ectoderm, endoderm and mesoderm).

Stem cells are important in regenerative medicine (a major and promising source of interest for successfully manufacturing organs), modeling, notably of diseases, and pharmacological screening.

Pluripotent stem cells appear to be a possible alternative due to their unlimited proliferation property and their differentiation capacity, allowing all the cell types of interest to be obtained in large amounts from the same donor. Furthermore, pluripotent stem cells make it possible to obtain a homogeneous population of differentiated cells, unlike primary cultures (heterogeneity, limit in number, risk of senescence).

The present invention is directed toward a method for differentiating human pluripotent stem cells into fibroblasts, preferentially into fibroblasts of connective tissues underlying an epithelium, and even more preferentially into dermal fibroblasts, under “clinical” conditions. The term “clinical” conditions means conditions under which the products used are manufactured in accordance with Good Manufacturing Practices (GMP) and can therefore be used in clinical studies, unlike “research”-grade products, which can only be used for research purposes.

Fibroblasts are “support” cells, which are notably present in connective tissue and are responsible for the synthesis of the extracellular matrix and collagen fibers (intercellular substance of connective tissue).

Within the meaning of the present invention, the term “connective tissues underlying an epithelium” means dense connective tissues with a predominance of collagen fibers, which play a mechanical role in the body, for instance the dermis, tendons or the cornea.

Dermal fibroblasts are of two types depending on their location in the dermis.

In its superficial part in contact with the epidermis (papillary dermis), the dermis is made up of oriented dense fibrous connective tissue and mainly contains papillary fibroblasts, whereas it is made up of non-oriented dense fibrous connective tissue, mainly composed of reticular fibroblasts in the deep dermis (or reticular dermis).

The method according to the present invention makes it possible preferentially to obtain dermal fibroblasts.

Advantageously, the fibroblasts obtained according to the method of the present invention are papillary dermal fibroblasts.

The “GMP” standards are a quality assurance concept established by the European (or American) Commission in the context of the manufacture of medicaments for human or veterinary use (EUDRALEX in France or FDA in the USA). They were created to limit the risks of cross-contamination of products, by insisting on hygiene practices, and also the risks of confusion: labeling/identification.

The principles of GMP require the writing of operating procedures and instructions that enable consistent quality production with compliant traceability. They also integrate processes, product quality and personnel safety. GMPs are currently organized in three parts:
Good Manufacturing Practice for medicaments for human use;
Good Manufacturing Practices for active substances used as starting materials in medicaments;
Good Manufacturing Practice documents giving recommendations regarding international requirements for batch certification.

Thus, in order to establish “clinical” differentiation protocols, the starting materials must meet the same GMP standards. The products used are therefore fully defined in terms of their compositions, concentrations, origins and sterility levels.

To date, there is no method for differentiating human pluripotent stem cells into fibroblasts which meets GMP standards, enabling the production of robust homogeneous specific cell populations (which do not deviate, remain the same over time).

There is thus a need to develop methods for obtaining human fibroblast populations that can be used in tissue therapy or for in vitro and in vivo models of human skin. Embryonic stem cells and somatic cells that are genetically reprogrammed so as to reproduce all the characteristics of embryonic stem cells (for instance “iPS”, meaning “induced pluripotent stem” cells) are extensively proliferating pluripotent stem cells and consequently have great potential in research and medicine. Several attempts at obtaining human fibroblasts from pluripotent stem cells have thus been described in the prior art.

Notably, the process described in the journal Stem Cell Research and Therapy by the team of Shamis et al. in 2011 uses human embryonic stem cells (hESC) cultured on a layer of mouse embryo-derived feeder fibroblasts inactivated by treatment with 4% para-formaldehyde (called “feeder”). (Shamis Y., Hewitt K. J., Carlson M. W., Margvelashvilli M., Dong S., Kuo C. K., Daheron L., Egles C., Garlick J. A. Fibroblasts derived from human embryonic stem cells direct development and repair of 3D human skin equivalents. Stem Cell Res. Ther. 2011 Feb. 21; 2(1):10. doi: 10.1186/scrt51).

This requires treating the feeder layer, for example 3T3 murine fibroblasts, with an agent which totally inhibits the proliferation (antimitotic) of these cells in favor of hPSC cells. However, the products used for blocking the proliferation of feeders may prove to be toxic to the cells of interest.

Furthermore, the feeders are of murine origin. In order for this system to be used with murine feeders, it must be ensured that these feeders can be removed during the process to purify the human fibroblast population. Since the feeders are also fibroblasts, it is not possible at present to separate human fibroblasts from murine fibroblasts.

The method described in Generation of 3D skin equivalents fully reconstituted from human induced pluripotent stem cells (iPSCs) by Munenari Itoh, Noriko Umegaki-Arao, Zongyou Guo, Liang Liu, Claire A. Higgins and Angela M. Christiano relates, for its part, to a culture system in suspension which requires treatment on D8 to make the cells adherent.

Thus, there is a need to improve the existing methods for generating and obtaining a homogeneous population of fibroblasts. Furthermore, there is also a need to obtain cells of greater purity.

The present invention proposes methods for producing iPSC- or ES-derived fibroblasts required for EB-independent clinical, research or therapeutic applications, which do not use feeder cells, and above all which provide a homogeneous and pure population of fibroblasts.

The present invention is directed toward a method for differentiating pluripotent stem cells into fibroblasts under “clinical” conditions.

### DESCRIPTION OF THE INVENTION

The present invention relates to a method for differentiating human pluripotent stem cells of embryonic or induced origin into fibroblasts, preferably into fibroblasts of the underlying connective tissue of an epithelium, said method being compatible with GMP standards.

More particularly, the present invention relates to a process for differentiating fibroblasts from human pluripotent stem cells, characterized in that the human pluripotent stem cells are cultured on an adherent system in the presence of a medium that is suitable for culturing fibroblasts and in the absence of feeder cells.

Advantageously, the method according to the present document is performed exclusively on an adherent system, i.e. it does not include a step of culturing in suspension. The absence of a step of culturing in suspension saves time and allows the entire process to be performed with the same culture medium. The methods of the prior art, which require a first step in suspension before the step on an adherent system, require the use of several different culture media for these various steps.

Moreover, the absence of feeder cells in the method according to the invention notably has the following advantages:

only one cell type is used, which allows a homogeneous population to be obtained without the need for a feeder cell removal step;

the use of feeder cells requires the use of an antimitotic agent and involves a step of checking that the cell cycle of said feeder cells has indeed stopped;

since the feeder cells may be of animal or human origin, their use presents a risk of contamination of the fibroblasts by the feeder cells.

The absence of feeder cells thus represents a significant advantage for the safety of the product obtained according to the method of the invention.

Advantageously, the medium that is suitable for culturing fibroblasts is a chemically defined medium which does not contain any additive of animal origin not controlled by Good Manufacturing Practice (or GMP) standards.

The term “adherent system” means any system that allows cells to adhere in 2D. Such a system allows a selection of homogeneous differentiated cells, unlike suspension systems which increase the risk of having heterogeneous populations. It is a surface covered with a matrix or coating. Preferably, the matrix used in the process according to the invention is a defined protein matrix. Preferably, the matrix is chosen from the group consisting of Matrigel™, L7 Coating™, laminin and vitronectin. In a particularly preferred manner, the matrix is the L7™ matrix sold by the company Lonza under the reference FP-5020.

The term “hPSCs” refers to any precursor cell of human origin which is capable of forming any adult cell. These cells are true cell lines in that they: (i) are capable of extensive proliferation in vitro in an undifferentiated state; and (ii) are capable of differentiating into derivatives of the three embryonic germ layers (endoderm, mesoderm and ectoderm) even after prolonged culturing. Human embryonic stem cells (hESC) are derived from fertilized embryos that are less than one week old (in the cleaving or blastocyte stage) or produced by artificial means (such as by nuclear transfer) which have equivalent characteristics. Other hPSCs include, but are not limited to, multipotent adult progenitor (MAP) cells, induced pluripotent stem cells (iPSC) and amniotic fluid stem cells.

In a preferred embodiment, the human pluripotent stem cells are obtained via methods that do not require the destruction of embryos.

In one embodiment, the pluripotent stem cells are human embryonic stem cells (“hESC”). Many hESC lines exist, including the RC-9 line (“Roslin Cells 9”), which was developed as a clinical-grade hESC line.

Alternatively, the hESCs or human iPSC cells may be selected from master cell banks that may be established for therapeutic purposes. Preferably, the hESCs or human iPSC cells may be selected to avoid or limit immune rejection in the majority of the human population. In general, hESCs or human iPSC cells are HLA homozygous for the genes encoding the major histocompatibility antigens A, B and DR, which means that they have a simple genetic profile in the HLA repertoire. The cells could be used to create a stem cell bank as a renewable source of cells that is suitable for preparing human skin substitutes intended for use in cell therapy of pathologies associated with skin damage (e.g. wound, burn, radiation, disease-related epidermal abnormalities). In another particular embodiment, the human pluripotent stem cells may bear a mutation or a plurality of mutations that are responsible for a genetic disease of the human skin.

The “induced pluripotent stem cells” of the present invention are cells induced to have pluripotency by reprogramming a somatic cell via a known and similar process. More specifically, a cell induced to have pluripotency can be obtained by reprogramming differentiated somatic cells such as fibroblasts, peripheral blood mononuclear cells and the like by expressing a combination of a plurality of genes selected from the group consisting of reprogramming genes, including Oct3/4, Sox2; mention may be made of Klf4, Myc (c-Myc, N-Myc, L-Myc), Glis1, Nanog, Sall4, lin28, Esrrb and the like. Examples of preferable combinations of reprogramming factors include (1) Oct3/4, Sox2, Klf4 and Myc (c-Myc or L-Myc) and (2) Oct3/4, Sox2, Klf4, Lin28 and L-Myc.

Induced pluripotent stem cells were obtained by Yamanaka et al. from a mouse cell in 2006. In 2007, induced pluripotent stem cells were also obtained from human fibroblasts and these have pluripotency and self-renewal competence similar to that of embryonic stem cells.

The term “differentiation”, as used herein, refers to a process via which an unspecialized or relatively less specialized cell becomes relatively more specialized. In the context of cell ontogeny, the adjective “differentiated” is a relative term. Thus, a “differentiated cell” is a cell that has progressed further along a certain developmental pathway than the cell with which it is compared. A relatively more specialized cell may differ from an unspecialized or relatively less specialized cell in one or more demonstrable phenotypic characteristics, for instance the presence, absence or level of expression of particular cellular components or products, for example RNA, proteins or other substances, the activity of certain biochemical pathways, morphological appearance, proliferative capacity and/or kinetics, differentiation potential and/or response to differentiation signals, etc., where such characteristics indicate the progression of differentiation towards the relatively more specialized cell. The process of differentiation starting with pluripotent stem cells generally involves a first step, known as induction of differentiation, in which the cells enter a differentiation pathway to give progenitors (at different stages of differentiation), followed by a second step of “maturation”, in which differentiation continues, resulting in a population of fully differentiated cells.

According to one embodiment of the invention, the pluripotent stem cells are donor-specific hiPSCs obtained by reprogramming peripheral blood mononuclear cells of said donor. Protocols for reprogramming donor peripheral blood mononuclear cells or CD34+ cells isolated from umbilical cord blood are known to those skilled in the art.

The term “medium that is suitable for culturing fibroblasts” or “fibroblast culture medium” means any medium which contains nutrients and factors allowing the in vitro culturing of fibroblasts. Typically, such a medium may include amino acids, minerals and trace elements (including selenium, manganese and zinc), vitamins (from the B group, notably folates, niacinamide and biotin), glucose, pyruvate, pH buffer and growth factors or cofactors (such as insulin, hydrocortisone, EGF (epidermal growth factor) and FGF (fibroblast growth factor)).

Preferably, the medium that is suitable for culturing fibroblasts comprises insulin, hydrocortisone, EGF and FGF.

Preferably, the medium that is suitable for culturing fibroblasts is supplemented with serum so as to obtain a final serum concentration of between 2% and 10% (vol/vol), preferentially between 4% and 6% (vol/vol), even more preferably 5% (vol/vol). Typically, the serum used in the process of the invention is a clinical-grade, GMP-approved serum, and the medium supplemented with this serum is called a “High Certification” or “HC” medium.

A particularly preferred medium that is suitable for culturing fibroblasts is CnT-PR-F, sold by the company CELLnTEC. This medium was specifically designed for culturing fibroblasts. It conventionally comprises essential amino acids, minerals and trace elements (including selenium, manganese and zinc), vitamins (from the B group, notably folates, niacinamide and biotin), glucose, pyruvate, and a pH buffer, but also contains, in addition to conventional media, insulin, hydrocortisone, and the growth factors EGF and FGF.

In one embodiment of the invention, the medium that is suitable for culturing fibroblasts is not the NHK medium (“Normal Human Keratinocyte medium”), described in Hewitt et al. 2009 and in Example 2.

In one embodiment of the invention, the medium that is suitable for culturing fibroblasts is not the SCES medium (“Serum-containing Embryonic Stem cell medium”) described in Hewitt et al. 2009 and in Example 2.

Hewitt K., Shamis Y., Carlson M., Aberdam E., Aberdam D., Garlick J. Three-dimensional epithelial tissues generated from human embryonic stem cells. Tissue Eng. Part A. 2009 15(11): 3417-3426.

More particularly, the present invention relates to a method for preparing fibroblasts derived from human pluripotent stem cells, comprising the steps of:

(a) optionally, forming and culturing aggregates or clusters of said pluripotent stem cells on an adherent system to support cell attachment and growth in the presence of a medium that is suitable for culturing human pluripotent stem cells;
(b) culturing the human pluripotent stem cells or adherent aggregates or clusters of said pluripotent stem cells on a cell culture surface coated with a defined protein matrix coating in the presence of a medium that is suitable for culturing fibroblasts;
(c) differentiating the human pluripotent stem cells into fibroblasts by culturing on a protein matrix in the presence of a medium that is suitable for culturing fibroblasts, for 12 to 16 days, preferably 13 to 15 days, even more preferably 14 days;
d) optionally, maturing the fibroblasts obtained in step c) at least during passaging on a protein matrix in the presence of a medium that is suitable for maturation;
e) optionally, maturing the fibroblasts obtained in step d) during passaging in the presence of a medium that is suitable for maturation, with or without protein matrix;
f) selecting the cells and obtaining a homogeneous population of fibroblasts.

Preferably, the medium that is suitable for maturing the fibroblasts may be the same medium as the medium used in step c). Even more preferably, the medium that is suitable for maturing the fibroblasts is free of BMP-4.

During the optional step a) of the method according to the invention, the pluripotent stem cells are amplified to obtain a population that is sufficient for experimental or clinical purposes. During this amplification step, the cells form colonies. The cell passaging may involve either manual passaging (which involves the operator manually cutting the colonies into clumps or pieces with a needle) or enzymatic passaging (for example using an EDTA solution). For the purposes of the present invention, the term “passaging” or “cell passaging” refers to any cell culture technique that is well known to a person skilled in the art. For example, the term may refer to a technique which involves the steps of (1) releasing cells from a solid support or substrate and dissociating those cells, and (2) diluting the cells in a medium that is suitable for further cell proliferation. Cell passaging may also refer to removing a portion of the liquid medium containing cultured cells and adding liquid medium to the original culture vessel to dilute the cells and allow further cell proliferation. In addition, the cells may also be added to a new culture vessel that has been supplemented with a medium that is suitable for further cell proliferation.

In one embodiment of the invention, the differentiation step c) takes place for 12 to 16 days, preferably 13 to 15 days, even more preferably 14 days.

The medium that is suitable for culturing fibroblasts is supplemented with Bone Morphogenetic Protein 4 (“BMP-4”) during all or part of step b). Typically, the final concentration of BMP-4 is about 0.01 to 50 nM, preferably about 0.2 to 0.4 nM, even more preferably about 0.27 nM.

The BMP-4 protein is preferably a recombinant protein having the sequence of the human form of BMP-4, for example BMP-4 sold by the company Peprotech under the reference AF-120-05ET.

According to one embodiment, the medium is supplemented with BMP-4 from D1 to D6.

This supplementation may be performed by adding BMP-4 on D1, the culture medium not being renewed in the following days (a single “pulse”).

Alternatively, the supplementation may be performed by adding BMP-4 on D1 and D4 (two “pulses”).

Typically, BMP-4 is no longer present in the medium from D7 onwards.

On conclusion of step c), the fibroblasts or fibroblast precursors obtained are isolated by virtue of their adhesion properties so as to continue their maturation.

According to one embodiment of the invention, the fibroblasts or fibroblast precursors are isolated by “natural” selection. On conclusion of step c), the cells are seeded at a very high density (minimum of 50 000 cells/cm²) on a defined matrix to allow the differentiated fibroblasts to adhere and to be cultured at a sufficient density (minimum of 10 000 cells/cm²) to limit the cellular stress induced by culturing. The next day, the cells of interest are attached to the substrate, whereas the poorly differentiated or undifferentiated cells are not.

The present invention also relates to a population of fibroblasts, preferentially fibroblasts of connective tissue underlying an epithelium, in particular dermal fibroblasts, obtained directly via the process described above.

Advantageously, the fibroblast population thus obtained is homogeneous.

Advantageously, the method according to the invention makes it possible preferentially to obtain papillary fibroblasts.

Specifically, there are two types of fibroblasts in the dermis: papillary fibroblasts and reticular fibroblasts. Advantageously, the process according to the present invention makes it possible preferentially to obtain papillary fibroblasts. These fibroblasts are more proliferative and aid in the correct stratification of the epidermis. Thus, for dermal repair applications, it is advantageous to have a majority of papillary fibroblasts.

The term “papillary fibroblasts” refers to a population of cells expressing a majority of the papillary fibroblast marker podoplanin (PDPN). According to the invention, a population of cells is considered to express a majority of a given marker if at least 90%, preferably at least 95%, even more preferably at least 98% of the cells express a given marker. The expression of a given marker by a population of cells can be measured via any appropriate technique, such as FACS.

Advantageously, the fibroblasts obtained according to the method of the present invention have features similar to those of fetal fibroblasts and enable better wound healing than adult fibroblasts.

The present invention also relates to the use of the dermal fibroblast population for the preparation of a skin substitute.

### BRIEF DESCRIPTION OF THE FIGURES

Other features, details and advantages of the invention will become apparent on examining the appended Figures.

FIG. 1

FIG. 1 represents protocols for the differentiation of hESC cells into fibroblasts.

FIG. 2

FIG. 2 represents the protocol for differentiating hESC cells into fibroblasts according to the invention.

FIG. 3

FIG. 3 represents the subtypes of fibroblasts in the skin and the associated markers.

FIG. 4

FIG. 4 represents the quality control of hPSC-derived fibroblasts.

FIG. 5

FIG. 5 represents the comparison of FRC9 and of primary fibroblasts.

FIG. 6

FIG. 6 represents the differentiation into fibroblasts starting with F-1432 IPS.

FIG. 7

Western blotting of total proteins following the differentiation into myofibroblasts of fibroblasts obtained from iPSCs, adult fibroblasts, neonatal fibroblasts and fetal fibroblasts.

FIG. 8

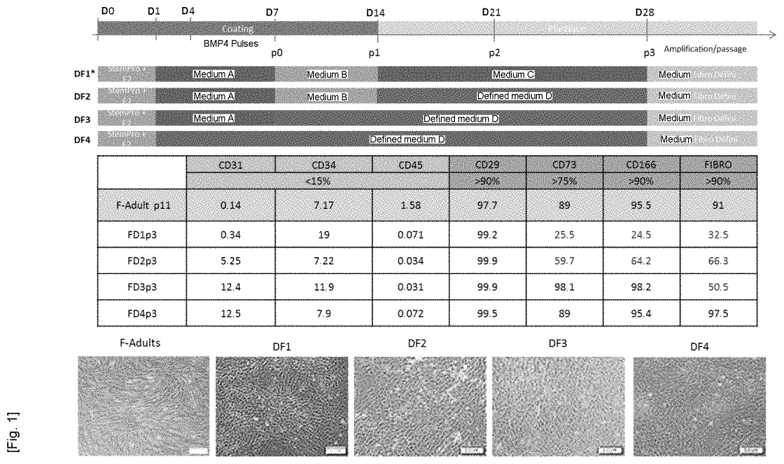

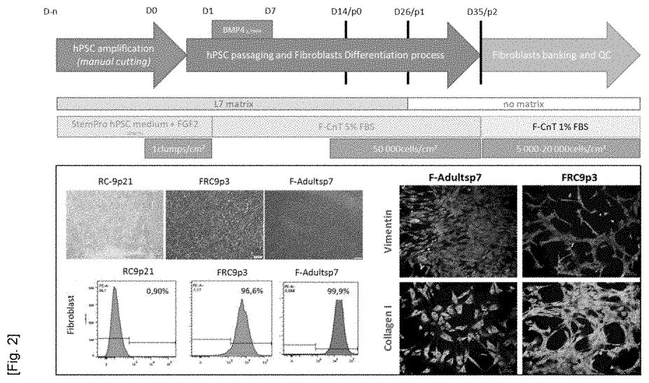

Immunofluorescence analysis of α-SMA-positive cells of cells differentiated into myofibroblasts.

### EXAMPLES

### Example 1: Materials and Methods

Pluripotent cell line used: hES line (RC9, Roslin Cells)

Primary fibroblast culturing:

The various types of primary fibroblasts used are:
Human Dermal Fibroblasts pooled foreskin: F-Adultes (HDFp, CELLnTEC)
Normal Human Dermal Fibroblasts Fetal Foreskin: HDFF (Cell Application, 106-05f)
Normal Human Dermal Fibroblasts neonatal Foreskin: HDFN (Promocell, C-12300)
Normal Human Dermal Fibroblasts adult: HDFA (Promocell, C-12302)

Clinical-grade differentiation protocol according to the invention:

Colonies of hPSC cells are cut with a needle and 1 clump/cm² is seeded in containers coated with L7 matrix in Stem Pro medium supplemented with stabilized FGF-2 for 24 hours (DO). The next day, the culture medium is changed to CnT-Prime-Fibroblast medium (F-CnT) supplemented with defined and secured fetal calf serum (final concentration: 5%) up to D14. BMP-4 treatment at 0.27 nM is applied on D1 and D4.

From D14/p0, when confluence is reached, the cells are passaged by trypsin treatment for 5 min at +37° C. The cells undergoing differentiation are reseeded at 50 000 cells per cm² up to D35/p2 on L7 matrix. The cells are thus ready to be used (banking, clinical, other uses).

### Example 2: Differentiation Protocol

Several differentiation conditions were tested and compared.

Condition 1 (FD1 or DF1) is performed with the same media as the protocol of Shamis et al. up to p3/D28, but without feeder and on the L7 matrix.

The media NHK and SCES are described in the publication by Hewitt K., Shamis Y., Carlson M., Aberdam E., Aberdam D., Garlick J. Three-dimensional epithelial tissues generated from human embryonic stem cells. Tissue Eng. Part A. 2009 15(11): 3417-3426.

The NHK medium comprises a mixture of DMEM:F12 3:1, 5% FCII, 0.18 mM adenine, 8 mM HEPES, 0.5 μg/mL hydrocortisone, 10⁻¹⁰ M cholera toxin, 10 ng/mL EGF and 5 μg/mL insulin.

The SCES medium comprises a mixture of DMEM:F12 1:1, 5% FCII and 1% nonessential amino acids.

Condition 2 (DF2 or FD2) is performed with the same media as the original protocol (DF1), i.e. the Shamis protocol but without feeder and on the L7 matrix, up to p1/D14 and then the F-CnT 5% medium up to p3/D28.

FD3 is performed with the same media as the original protocol up to p0/D7 and then 5% F-CnT medium up to p3/D28.

FD4: is performed solely with 5% F-CnT medium up to p3/D28.

On D21/p2, the cells of all the conditions are frozen and stored in nitrogen until the time of use.

For quality control, the cells are thawed and then amplified in F-CnT medium (without the addition of 4% serum as for differentiation) and amplified to 100% confluence. The morphology of the cells obtained is similar irrespective of the differentiation conditions FD1, FD2, FD3 and FD4 but are different than the primary cells (F-Adult). After passaging, the cells are analyzed by FACS so as to identify the populations obtained.

Several markers are analyzed: fibroblast characterization markers: CD29 (integrin p1), CD73/CD166 (mesenchymal stem cell markers) and “Fibro” (fibroblast markers).

Analysis of the expression of all these markers on primary adult cells (F-Adults) shows that the “fibroblast” profile is reflected by an expression of: CD29/CD166/FIBRO of greater than 90% and CD73 of greater than 75%.

Analysis of the four differentiation conditions FD1, FD2, FD3 and FD4 shows that only one differentiation reaches the specifications obtained with adult fibroblasts: the FD4 condition with an expression of CD29 at 99.5%, CD73 at 89%, CD166 at 95.4% and FIBRO at 97.5%.

This “FD4” condition is thus validated for the differentiation protocol. It is also the simplest since there is only one medium reference for the whole process, supplemented with 5% serum for the “differentiation” step between D1 and freezing at p2.

For the subsequent tests, the density was increased to 50 000 cells/cm² at the end of differentiation p0 and p1 and then from p2 onwards to between 5000-10 000 cells/cm². At 50 000 cells/cm² there are more adherent cells than at 5000-10 000 cells/cm² but the level of non-adherent cells is also higher. This differentiation end step (p0) thus appears to be a selection step for fibroblast “progenitors”. The sorting is done on the basis of adhesion: non-adhered cells are not sufficiently mature or are not fibroblast progenitors. This enables the future fibroblasts to be purified and the population to be made homogeneous in the course of the following passages.

These differentiation and passaging end densities made it possible to obtain hESC-derived fibroblasts proliferating to at least p7 (subsequent passages not tested) with no significant decrease in yield at each passage.

The established differentiation protocol thus consists of several steps:

On D0, colonies of hPSC cells are cut into clumps with a needle under a binocular loupe. Approximately 1 clump/cm² is seeded in stoppered containers (flasks) coated with L7 coating and in StemPro hESC medium with 10 ng/mL of stabilized FGF-2 (hPSC culture conditions) to allow hESC adhesion.

From D1 to D7, the StemPro/FGF2 medium is replaced with ready-to-use 1% F-CnT medium which is supplemented with an additional 4% FCS (to give a final concentration of 5%). When the same experiments were performed with 1% FCS, significant cell death was observed and the cells were less well differentiated into fibroblasts.

From D1 to D7, the 5% F-CnT medium is supplemented with BMP-4 (0.273 nM) to inhibit the neural pathway. Two pulses of BMP-4 are performed on D1 and D4. BMP-4 is present from D1 to D7 inclusive.

From D8 to D13, the cells are maintained with 5% F-CnT medium to allow proliferation and differentiation of the hPSCs.

On D14 (p0), the cells rinsed with PBS1× are placed in contact with 0.05% EDTA trypsin for 5-10 min. The trypsin action is stopped with 10% FCS and the cells are filtered through a 40 μm sieve to remove the undissociated cell clusters. The cells are then seeded at 50 000 cells/cm² in stoppered containers (flasks) coated with L7 coating and in 5% F-CnT medium.

The morphology of the FRC9 obtained after the RC-9 hESC differentiation process is very similar to that of the adult fibroblasts (F-Adult) and no longer resembles that of the starting RC-9 hESCs. Analysis of the expression of the FIBRO (or Fibroblast) marker shows an absence of expression of this marker on the RC-9 hESCs and an expression of 96.6% on the FRC9s, close to the level of the adult fibroblasts at 99.9%. In addition to this marker, the cells also express vimentin (fibroblast marker) and collagen I (secreted by mature fibroblasts) in the cytoplasm. Collagen I does not appear to be secreted by the FRC9 or F-Adults as there is no deposit. ELISA tests could be performed to observe the secretion of procollagen I.

### Example 3: Phenotypic Characterization of hPSC-Derived Reticular and Papillary Fibroblasts

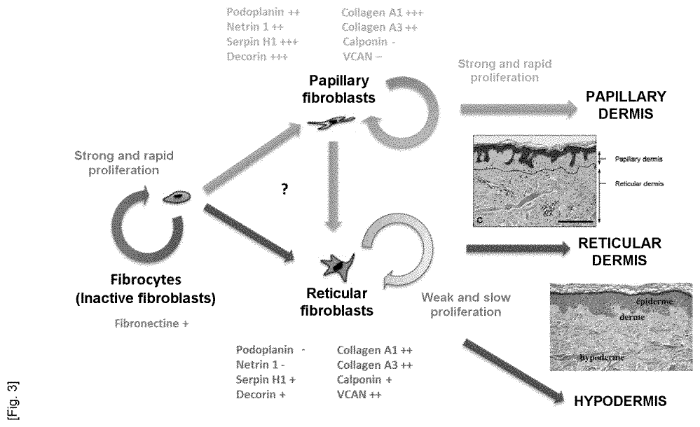

The literature shows that different types of fibroblasts exist and that, depending on their location within the same tissue or in a different tissue, they express specific markers (FIG. 3):

Fibronectin: involved in organizing the extracellular matrix (ECM)

Serpin H1 (HSP47): involved in collagen synthesis in dermal fibroblasts Decorin (DEC): involved in the assembly of collagen fibrils

Collagen I and III (COL1A and COL3A): ECM proteins

Podoplanin (PDPN): involved in ECM remodeling by papillary fibroblasts

Calponin 2 (CNN2): involved in inhibiting reticular fibroblast proliferation

Versican (VCAN): ECM protein secreted by reticular fibroblasts

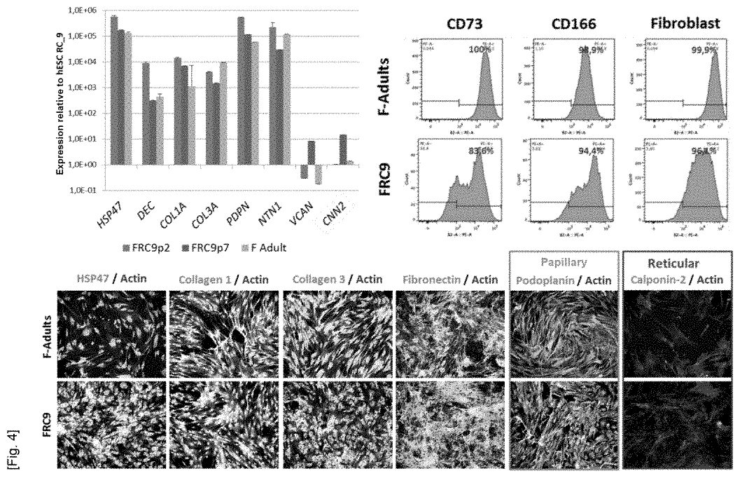

A wide range of markers from the literature was tested by qPCR, ICC and FACS to determine the exact identity of the fibroblast population produced by the differentiation protocol of the present invention (FIG. 4).

Gene expression analysis by qPCR (normalized on the 18S gene and RC-9 cells) shows that the RC9-derived fibroblasts express the markers HSP47, DEC, COL1A, COLA3, PDPN and NTN1 at high levels similar to those of adult fibroblasts. The reticular markers VCAN and CNN2 are not or are only sparingly expressed by FRC9p2 and adult fibroblasts. A slight increase in the expression of the reticular markers VCAN and CNN2 on FRC9p7 is observed.

FACS analysis shows that FRC9 and F-Adults express the CD73/CD166 markers at more than 80% and the “Fibroblast” marking at more than 96%.

Analysis by ICC shows that FRC9s and F-Adults have similar profiles:

HSP47 is indeed expressed in the endoplasmic reticulum,

Collagens I and III are also expressed in the cytoplasm with a location near the endoplasmic reticulum,

Fibronectin is expressed in the cytoplasm and there are deposits on the outside of the cells, which shows that the protein has been secreted,

Podoplanin is expressed throughout the cytoplasm,

Calponin 2 is expressed by very few cells.

The results show that the fibroblast differentiation protocol makes it possible to obtain dermal fibroblasts due to the expression of serpinH1/HSP47 and that the population obtained is mainly papillary due to the strong expression of podoplanin and the weak expression of calponin 2 which is only observed on a few cells. The F-Adults and FRC9s also express collagens I/III, but these proteins do not appear to be secreted since no deposits are detected, unlike fibronectin which is expressed and secreted. Collagen secretion by F-Adults and FRC9s appears to require different culture conditions.

### Example 4: Comparison of hPSC-Derived Fibroblasts and Primary Fibroblasts

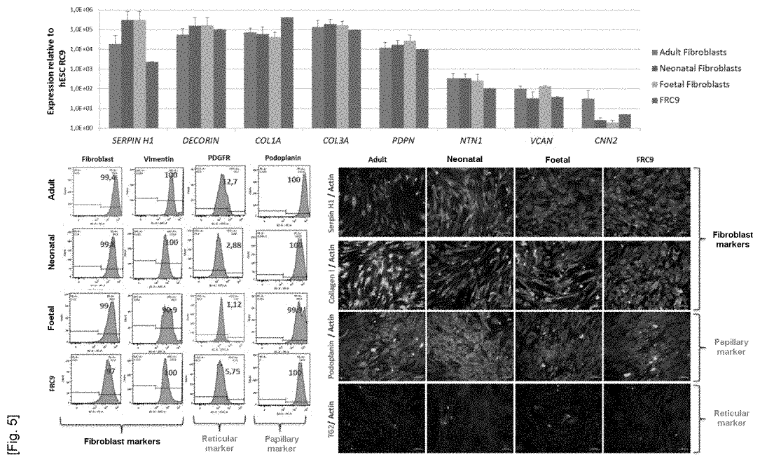

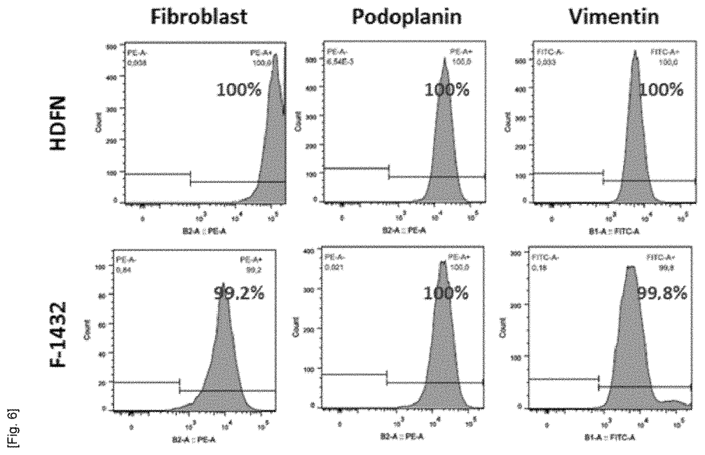

Still for the purpose of characterizing the cells and improving their quality control, a comparative study between FRC9 and three primary fibroblast lines (fetal, neonatal and adult) was performed (FIG. 5).

qPCR analysis shows that the expression profile of the DECORIN, COL1A, COL3A, PDPN, NTN1 and VCAN genes is similar between the three primary fibroblast types and FRC9. SERPIN H1 gene expression is similar to that of the adult cells but lower than that of the neonatal and fetal fibroblasts. The CNN2 gene is weakly expressed by the FRC9s and adult fibroblasts and is not expressed in the neonatal and fetal fibroblasts.

FACS analysis shows that the adult, neonatal and FRC9 fibroblasts have a similar profile for the markers “fibroblast”, vimentin and podoplanin with an expression of more than 97%. The fetal fibroblasts have a lower expression for vimentin marking with 90.9%. For the PDGFR marking (marking of reticular cells, involved in cell differentiation), the profile is globally observed to be weakly expressed, with an expression of less than 13% for all the lines.

Analysis by ICC shows that the three types of primary fibroblasts and FRC9 strongly and homogeneously express the markers Serpin H1, Collagen I, podoplanin with localization to the endoplasmic reticulum for the Serpin H1 and Collagen I markers. The TG2 marker (Transglutaminase 2, involved in the structure of the ECM), is very sparingly expressed by the four types of fibroblasts.

The analyses performed with the three types of fibroblasts do not make it possible to differentiate them and therefore to apply an “adult”, “neonatal” or “fetal” profile to the FRC9s.

### Example 5: Differentiation of iPSC Line PC1432 into Fibroblasts

The process used for the RC 9 line also works for iPS.

FACS analysis of iPS shows an expression profile similar to that of HDNF with more than 99% expression of the markers “Fibroblast”, podoplanin and vimentin.

The differentiation process thus makes it possible to obtain fibroblasts from iPS.

### Example 6: Characterization of Fibroblasts Derived from iPSC1432 Cells

The objectives of this study were, firstly, to evaluate the capacity of fibroblasts derived from iPSCs 1432 cells to differentiate into myofibroblasts after induction with TGF-beta and, secondly, to provide additional information regarding their characterization, notably on the developmental stage of these fibroblasts.

Indeed, obtaining early-stage fibroblasts would be advantageous because fetal and neonatal fibroblasts have faster and more efficient healing capacity than adult fibroblasts, notably for fetal fibroblasts for which skin repair in the early stages of gestation is rapid and without scarring.

To date, there are no specific markers for each stage (fetal, neonatal and adult) that allow them to be differentiated in vitro, but a comparative study of their respective functions can shed light on this matter. Publications have notably shown that fetal fibroblasts secrete much less TGF-beta 1 (which results in a weaker inflammatory response) than adult fibroblasts, but on the other hand that they do secrete much more collagen, notably type III and IV collagens (Larson, Longaker et al. 2010; Kishi, Okabe et al. 2011; Tang, Chen et al. 2014). The major difference between fetal and adult skin is the composition of the dermis (Coolen, Schouten et al. 2010). Where neonatal skin is histologically indistinguishable from adult skin, fetal skin shows an absence of elastin up to 22 weeks of gestation, but a larger amount of fibronectin. The fetal fibroblast has an intrinsic ability to synthesize a dermal extracellular matrix that is superior to that of adult fibroblasts, which makes it possible to generate a better-organized dermis at the sites of injury (more secreted matrix, but also more secreted metalloproteases for better remodeling).

In other studies, it was shown that proliferation did not differ between the neonatal and the adult fibroblasts, but their migratory potential was different (Mateu, Živicová et al. 2016). Furthermore, the authors show a larger pool of α-SMA (+) and Nestin (+) cells in the neonatal population compared to adults.

Differentiation into Myofibroblasts, Western Blotting (WB) Analysis: Comparison with Adult, Neonatal and Fetal Fibroblasts.

In this experiment, differentiation into myofibroblasts (induced by adding TGF-beta 1 to the culture medium) was performed in two different culture media: CnT-PR-F medium and DMEM 10% FCS medium, which is the medium conventionally used in the literature for performing this type of experiment. At the end of the differentiation, the total proteins of each condition were collected and analyzed by WB. The α-SMA protein is the best known marker of myofibroblasts. It is overexpressed when fibroblasts differentiate. Another feature of these myofibroblasts is an increase in cell contractility through myosin activation characterized by serine 19 phosphorylation. This specific form is also analyzed in this WB (the term P-MLC meaning phospho Myosin Light Chain).

Primary fibroblasts (adult and neonatal) differentiate into myofibroblasts only under the DMEM 10% FCS condition. The CnT-PR-F medium does not allow differentiation of these primary fibroblasts. Fibroblasts derived from iPSCs 1432 (FIBs IPSCs or FIBs 1432) and fetal fibroblasts are able to differentiate in both media, but with stronger differentiation in DMEM medium (weak increase in SMA expression in CnT-PR-F+TGF-beta medium). On the other hand, the DMEM 10% FCS medium appears to induce a basic differentiation of iPSCs and primary FIBs compared with CnT-PR-F medium independently of TGF-beta.

The increase in phospho-MLC2 after TGF-beta treatment is clearly visible by WB. Compared with α-SMA, it is visible for all the lines irrespective of the culture medium used (small increase for adults in CnT-PR-F but visible after quantification).

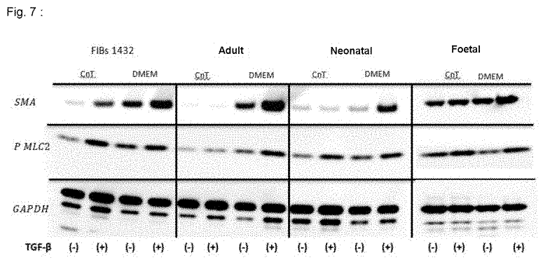

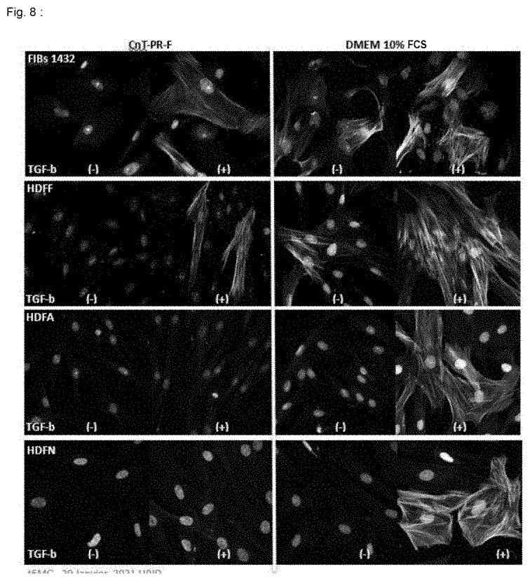

Overall, for the marker of contractility (P-MLC2) and that of the presence of myofibroblasts (SMA), it appears that the iPSC FIBs have a profile that is different from that of the adult and neonatal fibroblasts but similar to that of the fetal fibroblasts (cf. FIG. 7).

Differentiation into myofibroblasts, analysis by Immunofluorescence:

In parallel to the WB, the same differentiation experiment was performed for immunofluorescence analysis. The following images show the α-SMA-positive cells. The results follow those observed by WB, i.e. there are no α-SMA (+) cells in the CnT-PR-F+TGF-beta medium for adults and neonatals in contrast with iPSCs FIBs and fetal fibroblasts. In contrast, numerous SMA (+) cells are observed in DMEM+TGF-beta for the four lines. The adult and neonatal fibroblasts appear to be able to differentiate only in DMEM 10% FCS medium, whereas the 1432 FIBs and fetal fibroblasts are able to differentiate in both media as observed in WB.

On the basis of these experiments, it appears that 1432 FIBs have a differentiation profile into myofibroblasts that is similar to that of fetal fibroblasts.

1. A method for differentiating human pluripotent stem cells into fibroblasts, comprising the step of culturing the human pluripotent stem cells on an adherent system in the presence of a medium that is suitable for culturing fibroblasts and in the absence of feeder cells.

2. The method according to claim 1, wherein the fibroblasts are dermal fibroblasts.

3. The method according to claim 1, wherein the fibroblasts are at least 90% papillary fibroblasts.

4. The method according to claim 1, wherein the medium that is suitable for culturing fibroblasts comprises insulin, hydrocortisone, epidermal growth factor (EGF) and fibroblast growth factor (FGF).

5. The method according to claim 1, wherein the medium that is suitable for culturing fibroblasts is supplemented with Bone Morphogenetic Protein 4 (BMP-4).

6. A method for differentiating human pluripotent stem cells into fibroblasts comprising the steps of:
(a) optionally, forming and culturing aggregates or clusters of said human pluripotent stem cells on an adherent system to support cell attachment and growth in the presence of a medium that is suitable for culturing human pluripotent stem cells;
(b) culturing the human pluripotent stem cells or adherent aggregates or clusters of said human pluripotent stem cells on a cell culture surface coated with a defined protein matrix coating in the presence of a medium that is suitable for culturing fibroblasts;
(c) differentiating the human pluripotent stem cells into fibroblasts by culturing on a protein matrix in the presence of a medium that is suitable for culturing fibroblasts, for 12 to 16 days;
d) optionally, maturing the fibroblasts obtained in step c) at least during passaging on a protein matrix in the presence of a medium that is suitable for maturation;
e) optionally, maturing the fibroblasts obtained in step d) during passaging in the presence of a medium that is suitable for maturation, with or without the protein matrix; and
f) selecting cells obtained at step (c), (d) or (e) to obtain a homogeneous population of fibroblasts.

7. The method according to claim 6, in which step b) of amplifying the human pluripotent stem cells comprises manually passaging the human pluripotent stem cells.

8. The method to claim 6, in which step b) of amplifying the human pluripotent stem cells comprises enzymatically passaging the human pluripotent stem cells.

9. The method according to claim 6, wherein the medium that is suitable for culturing fibroblasts is supplemented with Bone Morphogenetic Protein 4 (BMP-4) at a concentration of between 0.1 and 0.5 nM.

10. A method for preparing a connective tissue, comprising culturing fibroblasts obtained according to the method of claim 1 under conditions suitable for the formation of connective tissue.

11. A method for the evaluation of compounds comprising the step of contacting the fibroblasts obtained according to the method of claim 1 under cell culture conditions suitable for the evaluation of the compounds.

12. A method for producing proteins for therapeutic use comprising the step of culturing the fibroblasts obtained according to the method of claim 1 under cell culture conditions suitable for production of the proteins.

13. The method of claim 6 wherein step c) is performed for 13 to 15 days.

14. The method of claim 6 wherein step c) is performed for 14 days.

15. The method according to claim 6, in which Bone Morphogenetic Protein-4 (BMP-4) is added at a concentration of between 0.2 and 0.4 nM on D1 and D4.

16. The method according to claim 6, in which Bone Morphogenetic Protein-4 (BMP-4) is added at a concentration of about 0.27 nM on D1 and D4.

17. The method of claim 10, wherein the connective tissue is dermis.

18. The method of claim 10, wherein the connective tissue is 3D skin.
